(19) United States
(12) Patent Application Publication
(10) Pub. No.: US 2015/0038578 A1
(43) Pub. Date: Feb. 5, 2015

(54) METHODS AND APPARATUSES FOR SURFACE FUNCTIONALIZATION AND COATING OF NANOCRYSTALS

(71) Applicant: Babak Nikoobakht, Potomac, MD (US)

(72) Inventor: Babak Nikoobakht, Potomac, MD (US)

(21) Appl. No.: 14/450,287

(22) Filed: Aug. 4, 2014

Related U.S. Application Data
(60) Provisional application No. 61/862,042, filed on Aug. 4, 2013.

(51) Int. Cl. A61K 47/48

(52) U.S. Cl. CPC A61K 47/48007
USPC 514/495; 556/110

(57) ABSTRACT

The present disclosure describes a method and series of compositions for surface functionalization of nanoparticles that eliminates the possibility of ligand loss in alien environments. In particular, the present disclosure provides method and compositions for production of highly-stable surface-functionalized nanorods of noble metals for in-vivo applications as well as use in composite materials.

### CROSS-REFERENCE TO RELATED APPLICATION

The present application claims priority to U.S. Provisional Application Ser. No. 61/862,042, filed Aug. 4, 2013, the disclosure of which is hereby incorporated by references in its entirety. This application relates to U.S. patent application Ser. No. 61/486,329, filed May 16, 2011 and U.S. Pat. No. 8,449,673 issued May 28, 2013, the disclosure of each of which is hereby incorporated by references in its entirety.

### FIELD OF THE INVENTION

The present disclosure generally relates to nanomaterials, and more particularly relates to methods and devices for production of surface-functionalized metal or semiconductor nanocrystals such as nanorods for in-vivo applications such as photothermal therapy and large scale production of composite materials such as fabrics and metamaterials.

### BACKGROUND OF THE INVENTION

Nanoparticles made of metals, semiconductors, or oxides are of interest for their mechanical, electrical, magnetic, optical, chemical and other properties. Noble metal nanostructures are of much interest because of their unique properties, including large optical field enhancements resulting in the strong scattering and absorption of light. The fields that are being impacted by the advancement in nanostructured materials include different areas such as electronics, materials, biology, medicine and other branches of physical sciences. To this end and as an example, gold nanoparticles are one of the most widely used classes of nanomaterials for chemical, bioanalytical, biomedical, electronics, optical and nanotechnological applications. Although there are numerous methods known for the synthesis of gold nanoparticles, the ability to change/control the coating and surface functionalization of gold nanoparticles on a large scale while preserving their aspect ratio, shape, monodispersity and optical properties remains challenging and one of the main areas of research.

### BRIEF SUMMARY OF THE INVENTION

Disclosed herein is a method comprising: covalently binding a first layer of ligands directly to a surface of a solid, wherein the first layer of ligands comprises first ligands with at least a first functional group and a second functional group, wherein the first layer of ligands comprises covalent bonds to the surface through the first functional group, wherein the first layer of ligands comprises cross-linked chemical bonds; and covalently binding a second layer of ligands to the first layer of ligands through the second functional group.

According to an embodiment, the first ligands comprise functional groups comprising atoms with one or more electron lone pairs.

According to an embodiment, the first functional group is selected from a group consisting of thiol, amine, hydroxyl, phosphine, phosphine oxides and a combination thereof.

According to an embodiment, the first layer of ligands comprises one or more two-dimensional ligands.

According to an embodiment, the first layer of ligands comprises dendrimers.

According to an embodiment, the first layer of ligands comprises amine or acrylate functional groups or a combination thereof.

According to an embodiment, the first layer ligands further comprises a third functional group, wherein at least two of the first, second and third functional groups are coplanar.

According to an embodiment, the second layer is bound to the first layer through a covalent bond, wherein the covalent bond is a C—N or C—C bond, or a combination thereof.

According to an embodiment, the binding the first layer to the surface and the binding the second layer to the first layer comprise performing the binding the first layer to the surface and the binding the second layer to the first layer in water.

According to an embodiment, the binding the first layer to the surface and the binding the second layer to the first layer comprise performing the binding the first layer to the surface and the binding the second layer to the first layer in an aqueous solution.

The method of claim 1, wherein the nanoparticle is a nanorod, or wherein the solid is a noble metal or a semiconductor.

Disclosed herein is a device comprising: a first layer of ligands directly and covalently bound to a surface of a solid; wherein the first layer of ligands comprises first ligands with at least a first functional group and a second functional group; wherein the first layer of ligands comprises covalent bonds to the surface through the first functional group; wherein a second layer of ligands comprises covalent bonds to the first layer of ligands through the second functional group; and wherein the first layer of ligands comprises cross-links.

According to an embodiment, the first ligands comprise functional groups comprising atoms with one or more electron lone pairs.

According to an embodiment, the first functional group is selected from a group consisting of thiol, amine, hydroxyl, phosphine, phosphine oxides and a combination thereof.

According to an embodiment, the first layer of ligands comprises one or more two-dimensional ligands.

According to an embodiment, the first layer ligands comprises amine or acrylate functional groups or a combination thereof.

According to an embodiment, the first layer ligands further comprises a third functional group, wherein at least two of the first, second and third functional groups are coplanar.

According to an embodiment, the second layer is bound to the first layer through a covalent bond, wherein the covalent bond is a C—N or C—C bond, or a combination thereof.

According to an embodiment, the nanoparticle is a nanorod, or wherein the solid is a noble metal or a semiconductor.

### BRIEF DESCRIPTION OF THE DRAWINGS

FIG. 1 shows a schematic of a surface of gold nanorods functionalized using a method or apparatus according to an embodiment.

FIG. 2A schematically depicts structure of ligands of stabilizing layer, where free binding sites of the ligands are shown as open locks.

FIG. 2B shows that upon introduction of the ligands to a surface of a nanorod, some of the biding sites become bound to surface.

FIG. 2C shows that a second layer of ligands may bind to the unbound binding sites of ligands in the stabilizing layer.

FIG. 3A shows a “Michael” addition reaction between amine and acrylate groups resulting in a C—N bond.

FIG. 3B shows covalent binding of the first layer ligands to the second layer ligands. In one embodiment this reaction is a Michael addition between amine and acrylate groups of the first and second layer, respectively.

FIG. 4 shows that a second layer of ligands including 2D- or 3D-ligands may be introduced.

FIG. 5 shows that the second layer of ligands may include multi-functional acrylate ligands.

FIG. 6A shows energy-dispersive X-ray spectroscopy of gold nanorods with only CTAB coated on their surface.

FIG. 6B energy-dispersive X-ray spectroscopy of gold nanorods where CTAB is substantially replaced by the stabilizing layer and the second layer of ligands.

FIG. 7 shows blue and red shifts in LSPR of gold nanorods with the stabilizing layer and the second layer of ligands.

FIG. 8A and FIG. 8B show an example of gold nanorods coated with a bilayer design including a stabilizing layer 205 with amine-termination and a second layer of PEG ligands with MW of .

### DETAILED DESCRIPTION OF THE INVENTION

In the present disclosure, reference is made to the accompanying drawings, which form a part hereof. In the drawings, similar symbols typically identify similar components, unless context dictates otherwise. Various embodiments described in the detailed description, drawings, and claims are illustrative and not meant to be limiting. Other embodiments may be used, and other changes may be made, without departing from the spirit or scope of the subject matter presented herein. It will be understood that the aspects of the present disclosure, as generally described herein, and illustrated in the Figures, can be arranged, substituted, combined, separated, and designed in a wide variety of different configurations, all of which are contemplated herein.

One useful morphology of gold nanoparticles is rod-or cylindrical-shaped. Particles with this shape are called nanorods with typical dimensions ranging from 1-200 nm. Compared to other shapes of nanoparticles including spheres and shells, nanorods are more favorable for in-vivo applications due to their smaller size and tunable optical resonance in the near infra-red region (NIR). Moreover, their relative scattering to absorption contribution can be easily tuned by a change in their dimensions. Gold nanorods offer superior NIR absorption and scattering at much smaller particle sizes. Smaller sized nanorods also offer better cell uptake as compared to the larger nanoshells and nanospheres. This, in addition to the potential noncytotoxicity of the gold material, easy optical tunability, and facile synthesis, makes gold nanorods promising nanoparticle agents for use in biomedical diagnosis and photothermal therapy applications.

Nanorods may be characterized by their aspect ratios, which is defined as the length to the width of a nanorod. Their width typically range from about 3 nm to about 70 nm and their length is about 20 nm or more. Gold nanorods are also identified by their longitudinal surface plasmon absorption (LSPR) which is an absorption band tunable in the visible and NIR regions. Small scale synthesis methods may produce gold nanorods with aspect ratios less than 6. Gold nanorods with LSPR of 800 nm to 1000 nm are of interest in biotechnological applications due to their absorption in the NIR region of the spectrum and transparency of biological tissues in this region.

Gold nanorods as well as other plasmonic nanoparticles including shells, cubes, and cages may be useful to selectively destroy cancer cells via a photothermal effect and also function as contrast agents due to their strong light scattering. Challenges related to the surface quality of nanorods are associated with the particle's ability to stay in the so-called “stealth” mode when deployed in body. This means long circulation time (of at least 50% of the injected dose) in the affected area for therapy or imaging; absence of non-specific binding of nanoparticles to non-targeted cells and the absence of particle accumulation in body due to their aggregation. Nearly all these characteristics highlight one important specification and that is the ability to properly surface functionalize nanorods. This is a technological barrier for all shapes of nanoparticles and is not limited to gold nanorods. The surface coating of the as-prepared gold nanorods may be cetyltrimethylammonium bromide (CTAB). CTAB may be cytotoxic and to promote non-specific cellular uptake of nanorods [6]; accordingly, the focus of the scientific community has been on its replacement with benign ligands and specific functional groups. However, CTAB removal is shown to reduce the long life time of the nanorods. More importantly, the newly adsorbed ligands are likely to depart from the gold surface, rendering nanorods unstable for their designated tasks.

For CTAB-coated nanorods, there is a constant dynamic exchange of ligands between the gold surface and solution. If the surrounding nanorod solution does not have sufficient CTAB, the exchange process becomes a one-way reaction causing irreversible CTAB release to the surrounding solution. This has two negative implications for biological applications: cytotoxicity of free CTAB, and particle instability due to release of CTAB. The CTAB coating also negatively impacts the integration of CTAB-coated nanoparticles to other materials for manufacturing composite materials. For instance, these nanoparticles are only dispersible in water-based solutions, and in organic media, or non-polar solvents, they aggregate. Furthermore, CTAB-coated nanoparticles in water or liquid media do not survive below the freezing point temperatures and permanently aggregate.

Electrostatic adsorption of negatively charged polymer chains around CTAB-coated nanorods is a simple approach to conceal the positive charge of the CTAB ligands and also to improve its binding affinity to biomolecules such as antibodies. Another approach is the use of ligands that have stronger affinity toward gold, e.g., thiol-terminated ligands (such as HS-PEG) compared to the quaternary ammonium head group of the CTAB. A variety of long and short, single or double chain ligands containing N, O, S functional groups with electron lone pairs have been introduced to nanorods either in non-aqueous or aqueous solutions. Overnight or short periods of incubation and stirring help to desorb CTAB ligands and replace them with the new ligands. CTAB has a stronger binding affinity toward (110) facet, which is the energetic of all other facets including (100) and (111). As such in their replacement with new ligands, CTAB desorption from (110) facets are thought to be slower. Results also show that upon replacing CTAB with new ligands, shape stability of gold nanorods could degrade on a time scale of hours to days. This is because the newly adsorbed ligands are also likely to depart the gold surface.

This departure depends on the extent of new ligand's surface coverage and the applied coating method. To improve the ligand surface coverage of nanorods, thiolated analogue of the CTAB (16-mercaptohexadecyl)trimethylammonium bromide) may be used to replace the CTAB. A two-step ligand addition and exchange process has also been used, in which a short first ligand (4-Mercaptophenol) is introduced to nanorods to enable their miscibility with less polar solvents and thus more flexibility for organic reactions. Then the second ligand (carboxybiphenyl-terminated polystyrene) is covalently added to the first layer. This approach, although with a low yield, may be used for dispersion of gold nanorods in non-aqueous solvents. In another approach, CTAB may be exchanged with thiolated ligands via a two-phase extraction. In this process, first, CTAB is removed from nanorods by transfer into an organic phase of dodecanethiol ligands. The thiolated nanorods are then extracted into an aqueous phase by mercaptocarboxylic acids under heat of 70° C. to 95° C. Such processes suffer from multiple steps of chemical treatments with limited yield and a wide range of chemicals/solvents for processing, which make the overall process expensive and less adaptable for industrial applications. In another approach, amine dendrimers are first functionalized with thiol groups to create the binding sites to the gold surface and then are introduced to gold nanorods via a roundtrip-phase transfer process. During the phase transfer cycle, the gold nanorods are transferred from aqueous to organic phase and then to aqueous phase. The amine groups are not considered involved in binding to the gold surface and merely used for conversion to other functional groups such as carboxylic acid in the consequent steps.

Organic or inorganic shells around nanoparticles have also been proposed to potentially cover the remaining CTAB, thereby minimizing gradual loss of CTAB and maintaining the integrity of the nanorods. Examples include, the use of precipitation polymerization process to coat citrate-capped nanospheres with poly(N-isopropylacrylamide) (pNIPAm) nanogels, branched-PEG ligands containing amine groups and lipophilic moieties to form micellar shells around gold nanorods, and a two-photon photopolymerization process in which prepolymers surrounding gold nanospheres locally polymerize using a luminescence induced by multiphoton-absorption of the metal particle.

According to an embodiment, a method may change the surface functionalization of nanorods. According to an embodiment, an apparatus may functionalize surfaces of nanorods. According to an embodiment, the apparatus and method may allow high throughput coating with a built-in mechanism for securing the ligands on the gold surface via a cross-linked network of covalent bonds or polymer chains, and may lead to rational and scalable synthesis of highly surface-functionalized nanorods that have undetectable cytotoxicity, site specific binding and stability.

The disclosure is focused on gold nanorod surface functionalization as an exemplary embodiment. However, the method and apparatus disclosed herein may be adapted to functionalize other nanomaterials, examples of which include nanomaterials comprising other noble metals or semiconductors such as silver, copper, platinum, CdSe and the like. Also, whenever the disclosure refers to gold nanorods or gold ions, it is to be understood that other noble metals may take the place of gold.

As used in this application, the term “nanorod” is intended to include solid cylindrical objects less than 1000 nm in size. In this application, the following terms are meant to be synonymous with “nanorod”: rod shaped nanocrystals, cylindrical shaped nanocrystal, spheroidal shaped nanocrystal, and one dimensional nanocrystals. The term “nanocrystal” is intended to include crystals less than 1000 nm in size. The term “nanoparticle” is intended to include nanocrystals with different shapes with a size less than 400 nm.

According to an embodiment, a high throughput coating method includes securing ligands on nanoparticle's surface via a cross-linked network of covalent bonds or polymer chains. This method allows rational and scalable synthesis of highly surface-functionalized nanorods with reduced cytotoxicity, site specific binding and stability. This method also allows reduced ligand release from nanorod surfaces and acceptance of an outer layer of ligands covalently bound to the ligands directly attached to the nanorod surfaces. Examples of the ligands in the outer layer include polyethylene glycol for biotechnological applications or acrylate monomers/polymers for paints and surface coatings.

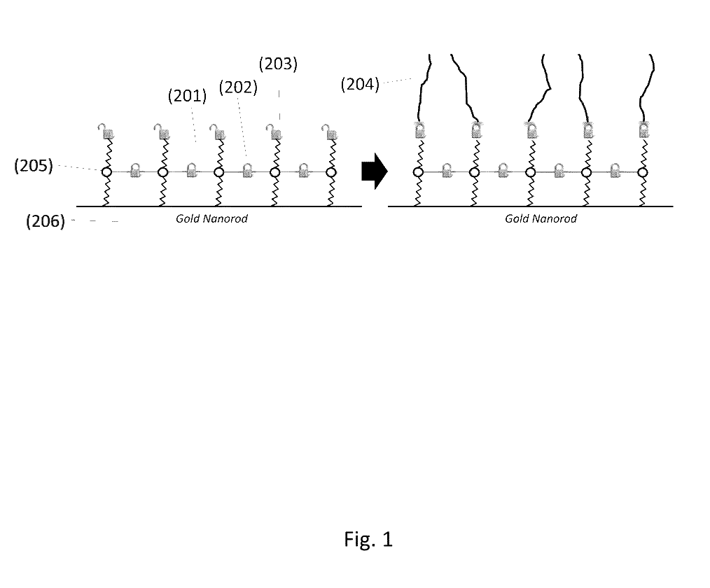

FIG. 1 shows a schematic of a surface of gold nanorods 206 functionalized using a method or apparatus according to an embodiment. The surface has a layer 205 of ligands directly attached thereto. The ligands in layer 205 (“stabilizing layer”) are interlocked, through the cross-linkage (depicted as closed locks) of organic functional groups 202. The cross-linkage reduces dissociation of the ligands in layer 205 from the nanorods 206. The ligands in layer 205 contain unreacted functional groups 203 (depicted as open locks) suitable for reaction with another layer of ligands 204. Ligands in layer 205 may be attached to the surface of the nanorods 206 by any suitable reactions. The existing reactions and surface functionalization methodologies heavily rely on securing the ligands on nanoparticles via the head group of the ligands. Currently, popular surface functionalization methods may use ligands with head groups such as thiol that bind to the surface of gold. Examples of these ligands include thiolated-polyethylene glycol (HS-PEG), thiolated-DNA and other head groups including amines (NH₂—). Merely using such head groups may not offer good control of the ligand coverage of the surface of nanorods and may have high dissociation of the ligands from the surface as there is no built-in mechanism to avoid their departure from the particle surface. High ligand dissociation may lead to instability and aggregation of the nanorods. Thiol chemistry as the most popular route does not guaranty the permanent stability of the ligands on the particle surface and introduces a high risk in stability of thiolated nanoparticles. This is an important roadblock for in-vivo applications or manufacturing of composite materials such as paints and nanoparticle-based coatings.

The extent of surface coverage of ligands may be affected by the strength of attachment of any existing surface groups (e.g., CTAB) on the nanorods. For example, high surface energy facets of nanorods, e.g., (110), have a strong binding with CTAB and thus accept the ligands slower and tend to have less surface coverage. Poor surface coverage may lead to short life time (e.g., degradation on a time scale of hours to days) of the nanorods.

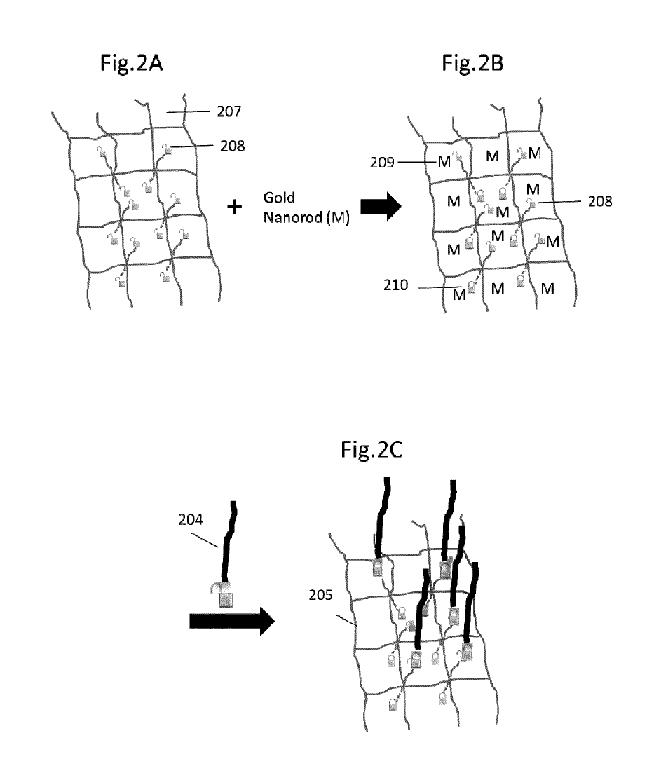

According to an embodiment, stabilizing layer 205 may include ligands 207 with multiple binding sites (“two-dimensional ligands,” “2D-ligands,” “three-dimensional ligands” or “3D-ligands”). Ligands 207 have reduced dissociation from the surface 209 of the nanorod because at each moment of time ligands 207 maintains several binding sites 210 to the surface 209. FIG. 2A schematically depicts structure of ligands 207, where free binding sites 208 of ligands 207 are shown as open locks. As shown in FIG. 2B, upon introduction of ligands 207 to a surface 209 of a nanorod, some of the binding sites become bound to surface 209. These bound binding sites 210 are depicted as closed locks. The remaining binding sites 208 are still unbound and may accept binding of other ligands.

Ligands 207 may have functional groups containing atoms such as N, S, P, O with electron lone pairs that can bind to empty orbitals of a metal or semiconductor. Examples of 2D-ligands include dendrimers such as poly(amidoamine) (also known as PAMAM). This large size ligand has tree-like branches and could be enlarged by adding more branching layers. As its size increases the molecule becomes curved and non-flat. Thus in higher generations of such dendrimers some of the amine binding sites could bind to nanoparticle surface and some at the center of the ligand may stay un-bound as shown in FIG. 2B. Examples of 2D-ligands also include monomer ligands with both amine and acrylate functional groups, such as poly(aminoethyl methacrylate-co-methyl methacrylate). 3D-ligands include molecules that have at least three functional groups that at least two of which reside in a hypothetical flat plane. Examples of 3D-ligands include proteins, peptides or synthetic polymers such as poly(N-isopropylacrylamide) (pNIPAm). In another aspect of this invention, 2Dligands include monomer ligands including both amine and acrylate functional groups such poly(aminoethyl methacrylate-co-methyl methacrylate).

In one embodiment, as shown in FIG. 2C, a second layer of ligands 204 may bind to the unbound binding sites 208.

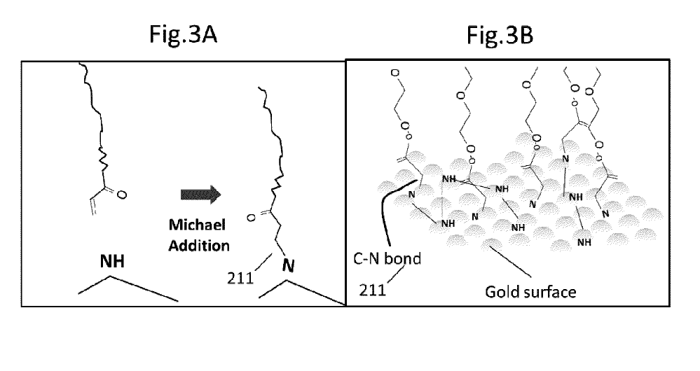

According to an embodiment, the bond between ligands 204 and ligands 207 may be a covalent bond such as a C—N bond formed by “Michael” addition between acrylate functional groups on ligands 204 and unbound amine groups of ligands 207. An example of an acrylate functional group is acrylate-terminated polyethylene glycol with molecular weights ranging about 1000 to 20000. An example of amine functional groups is poly(amidoamine) with molecular weights ranging about 1000 to 1000,000. A “Michael” addition reaction occurs between amine and acrylate groups resulting in a C—N bond 211 as shown in FIG. 3A.

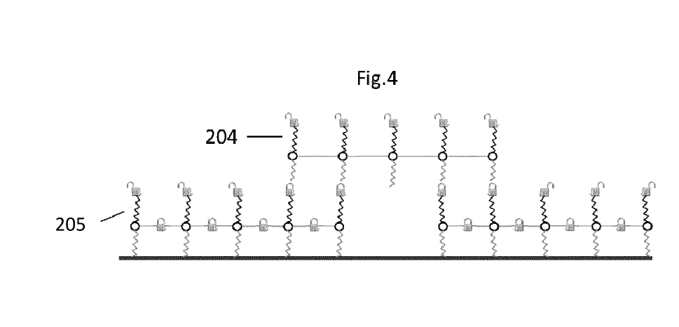

According to an embodiment, the bond between ligands 204 and ligands 207 may be amide bonds formed, for instance, by reaction between a carboxylic acid and amine groups Each 2D- or 3D-ligand 207 has a finite surface area of a few square nanometers. In one embodiment, a second layer of ligands 204 that include 2D- or 3D-ligands may be introduced, as shown in FIG. 4.

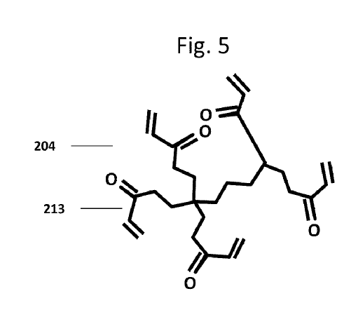

Ligands 204 in the second layer may include functional groups that can form covalent bonds with unbound functional groups of ligands in the stabilizing layer 205. In one embodiment these functional groups could be acrylate and amine groups. In an embodiment, as shown in FIG. 5, examples of ligands 204 may include multi-functional acrylate ligands 213 such as dipentaerythritol pentaacrylate, tris (2-hydroxy ethyl) isocyanurate triacrylate, or propoxylated (3) glyceryl triacrylate.

Such multi-functional ligand allows binding to the relevant chemical functions of ligands in the stabilizing layer 205 and connection to other incoming ligands such as long chain amine-terminated ligands.

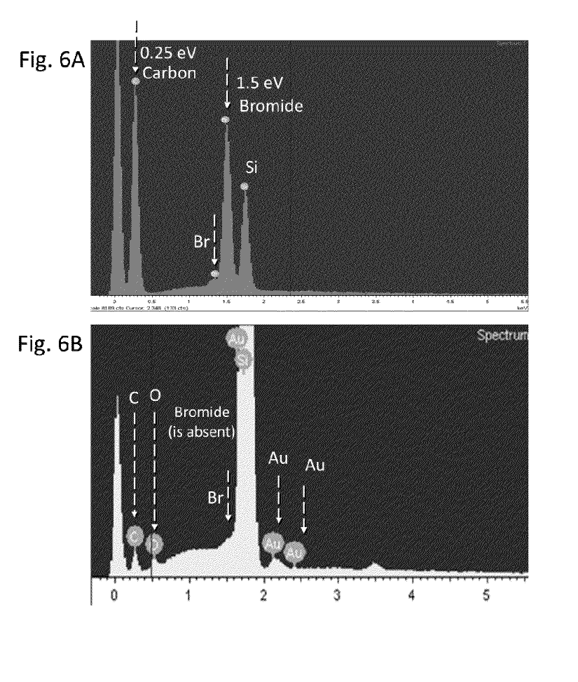

Surface characterization of gold nanorods in their as-prepared form,e.g., coated with CTAB is shown in FIG. 6A. The peaks characteristics of CTAB include the strong bromide and carbon peaks at 2.5 eV and 0.25 eV, respectively. The bromide is the counter ion associated with the positively charged quaternary ammonium head group of the CTAB. Carbon peak originates from the long chain of the CTAB ligand containing 16 carbon atoms. After removing the bound CTAB and binding of the stabilizing layer 205 and ligands 204 in the second layer, the signature peaks associated with C and Br elements reduce significantly, as shown in FIG. 6B.

According to an embodiment, all reaction steps (e.g., adding layers of ligands, cross-linking, etc.) are performed in water or an aqueous solution. In an embodiment, no phase transfer to an organic phase is required. The reactions between different layers of ligands can take place at room temperature and thus a suitable ambiance for biotechnological applications. For example, PEG layer is fabricated on gold nanorods by first binding an amine-terminated ligand as the stabilizing layer 205, by adding an aqueous solution of the amine-terminated ligand with a concentration range of 0.00001 M to 0.5M to CTAB-coated gold nanorods. Within about an hour, the second layer of ligands 204 that include a tri-functional acrylate ligand such as propoxylated (3) glyceryl triacrylate with a concentration range of about 0.00001 M to 0.001 M is introduced to the aqueous solution. After stirring the solution for at least about 20 minutes the second layer and the stabilizing layer link together via a series of Michael additions. The unreacted acrylate groups of this tri-functional acrylate ligand are used to covalently bind to short or long chain ligands with an amine terminal group. An example of a short chain ligand includes 6-aminocaproic acid and a long chain ligand is amine-terminated PEG.

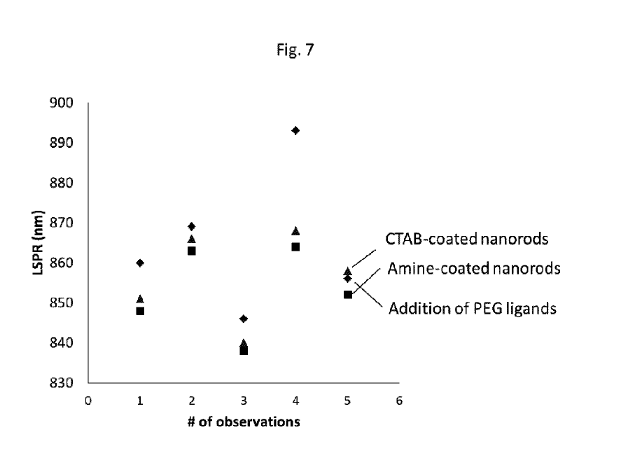

In another example, acrylate terminated ligands such as acrylate-terminated PEG are bound to the stabilizing layer by, for example, introducing an aqueous solution of short or long chain ligands with a concentration range of 0.00001M to 0.1 M introduced to the nanorods to form the C—N bond between these ligands and the stabilizing layer on nanorods. Optical spectroscopy can be used to monitor the progress in ligand binding to the nanorods. For instance, by adding an amine-terminated ligand the longitudinal surface plasmon resonance (LSPR) of CTAB-coated nanorods (FIG. 7, diamond dots) blueshifts to lower values (square dots). Binding the second layer results in blue or red shift depending on the structure of the ligands in that layer. For instance, FIG. 7 shows a redshift in the LSPR (triangle dots) in the case where very long PEG ligands with MW of 5000 are used as the second layer.

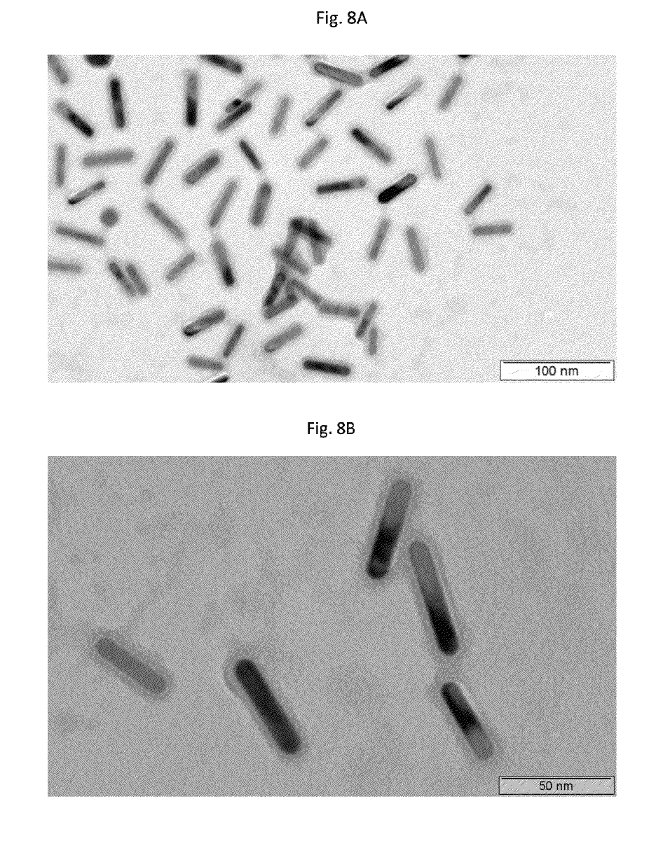

Transmission electron microscopy (TEM) shows the robustness of the disclosed techniques in coating gold nanorods. FIG. 8A and FIG. 8B show an example of gold nanorods coated with a bilayer design including a stabilizing layer 205 with amine-termination and a second layer of PEG ligands with MW of 5000.

According to an embodiment, the method disclosed herein may allow the nanorods to survive when their surrounding medium goes through phase changes between liquid and solid. The nanorod coated with the disclosed method may tolerate temperature below freezing point of the solution the nanorods are in. This is evident by comparing the absorption spectra of the nanorods before and after the freezing cycle.

The surface functionalized gold nanorods show physiochemical properties that are different from CTAB-coated nanorods. CTAB-coated nanorods have been proposed for in-vivo applications such as photothermal cancer therapy or as contrast agents for imaging; however, due to their surface coating they cannot be effective as they cause cytotoxic effects in a living organism. They also aggregate, which makes them less effective. In one aspect of this invention, the newly surface-functionalized nanorods may be injected via intravenous injection to blood or be used in combination with an ointment for external application to a surface including human skin or be used orally.

In one aspect of this disclosure, the stabile and robust surface coating allows long circulation time in body leading nanorods to cancer tumor. The robust coating also allows sufficient accumulation of nanorods around tumors within an optimum time period before excretion from body. Optimal accumulation is critical in rapid rise of temperature around the cancer tumor or diseased area. Optical excitation of LSPR of nanorods results in temperature rise of 55° C. to 70° C. in the tumor area resulting in tumor shrinkage and disappearance in a period of three to four weeks. Optical excitation in the range of 800 nm to 1100 nm with a power ranging from 0.2 Watt to 5 Watt may be used to excite a population of functionalized nanorods in the vicinity of a diseased area. For malignant or non-malignant tumors deeper inside body such as kidney, GI organ, prostate, bladder, gynecological organ, lung, brain and breast optical fibers and waveguides may be used for delivery of the optical excitation.

In another aspect of this invention, a method is disclosed for collective cross-linkage of nanoparticles. In this process, after linkage of the second layer ligands, when nanorods surface ligands are terminated with acrylate functional groups, a solution of multi-functional acrylate ligand and a photopolymerization initiator such as Lucirin TPO-L polis are introduced. In one aspect of this invention, using a UV light when the solution is forced through a planar channel transparent to the curing wavelength. The channel width ranges from 10 micrometer to 1 m, and its thickness ranges from 100 nm to 1 mm and its length ranges from 100 micrometer to 1 m. The UV-exposed nanorod solution is pumped out of the channel forming sheets of nanorod-based polymers with fill factors ranging from 2% to 98% fill factors.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A surface coating method for formation of stable nanoparticles for in-vivo applications and composite materials comprising:
covalently binding a first layer of ligands directly to a particle surface, wherein the first layer of ligands comprises first ligands with at least a first functional group and a second functional group, wherein the first layer of ligands comprises covalent bonds to the surface through the first functional group, wherein the first layer of ligands comprises cross-linked bonds; and
covalently binding a second layer of ligands to the first layer of ligands through the second functional group.

2. The method of claim 1, wherein the first layer ligands comprise functional groups comprising atoms with one or more electron lone pairs.

3. The method of claim 1, wherein the first layer functional groups is selected from a group consisting of thiol, amine, oxygen and a combination thereof.

4. The method of claim 1, wherein the first layer ligands comprise one or more two-dimensional ligands.

5. The method of claim 1, wherein the first layer of ligands comprise dendrimers.

6. The method of claim 1, wherein the first layer ligands comprise amine or acrylate functional groups or a combination thereof.

7. The method of claim 1, wherein the first layer ligands further comprise a third functional group, wherein at least two of the first, second and third functional groups are coplanar.

8. The method of claim 1, wherein the second layer is bound to the first layer through a covalent bond, wherein the covalent bond is a C—N or C—C bond, or a combination thereof.

9. The method of claim 1, wherein the binding the first layer to the surface and the binding the second layer to the first layer comprise performing the binding the first layer to the surface and the binding the second layer to the first layer in an aqueous medium.

10. The method of claim 1, wherein the particle is a nanorod, or wherein the solid is a noble metal or a semiconductor.

11. The method of claim 1, wherein the in-vivo application is treating cancer.

12. A method of treating cancer comprising administering to a subject in need thereof, comprising a composite of claim 1.

13. The method of claim 12, wherein surface-functionalized nanorods enhance the concentration of nanorods and light absorption around cancer cells.

14. A device for formation of stable nanoparticles for in-vivo applications and composite materials comprising: a first layer of ligands directly and covalently bound to a particle surface;
wherein the first layer of ligands comprises first ligands with at least a first functional group and a second functional group;
wherein the first layer of ligands comprises covalent bonds to the surface through the first functional group;
wherein the first layer of ligands comprises cross-linked bonds; and
wherein a second layer of ligands comprises covalent bonds to the first layer of ligands through the second functional group.

15. The device of claim 14, wherein the first ligands comprise functional groups comprising atoms with one or more electron lone pairs.

16. The device of claim 14, wherein the first functional group is selected from a group consisting of thiol, amine, oxygen and a combination thereof.

17. The device of claim 14, wherein the first layer of ligands comprise one or more dendrimer.

18. The device of claim 14, wherein the first layer of ligands comprise amine or acrylate functional groups or a combination thereof.

19. The device of claim 14, wherein the first ligands further comprise a third functional group, wherein at least two of the first, second and third functional groups are coplanar.

20. The device of claim 14, wherein the second layer is bound to the first layer through a covalent bond, wherein the covalent bond is a C—N or C—C bond, or a combination thereof.

21. The device of claim 14, wherein the solid is a nanoparticle, wherein the particle is a nanorod, or wherein the solid is a noble metal or a semiconductor.

22. The device of claim 14, wherein the in-vivo application is treating cancer using photothermal therapy.

23. A device of treating cancer comprising administering to a subject in need thereof, comprising a composite of claim 1.

24. The device of claim 23, wherein surface-functionalized nanorods enhance the concentration of nanorods and light absorption around cancer cells.
